(19) United States
(12) Patent Application Publication
(10) Pub. No.: US 2022/0373530 A1
(43) Pub. Date: Nov. 24, 2022

(54) Method And System For Detecting At Least One Contaminant In A Flow Of A Liquid Fuel

(71) Applicant: National University of Singapore, Singapore (SG)

(72) Inventors: Vivek Kolladikkal Premanadhan, Singapore (SG); Boon Chit So, Singapore (SG); Kin Cheong Lee, Singapore (SG)

(73) Assignee: National University of Singapore, Singapore (SG)

(21) Appl. No.: 17/623,199

(22) PCT Filed: Jun. 26, 2020
(86) PCT No.: PCT/SG2020/050373

§ 371 Date: Dec. 27, 2021

(30) Foreign Application Priority Data
Jul. 3, 2019 (SG) 10201906212T

(51) Int. Cl. G01N 33/22; G01N 11/04; G01N 9/32

(52) U.S. Cl. CPC G01N 33/22; G01N 11/04; G01N 9/32

(57) ABSTRACT

A method of detecting at least one contaminant in a flow of a liquid fuel includes measuring one or more parameters of a flow of the liquid fuel. Based on the measured one or more parameters, one or more properties of the liquid fuel are determined. A plurality of features are from selected ones of the one or more parameters and one or more properties. A trained classification model is applied on the extracted features to determine a type and a quantity of at least one contaminant in the liquid fuel.

### TECHNICAL FIELD

The present disclosure relates broadly, but not exclusively, to fuel quality monitoring, particular, methods and systems for detecting at least one contaminant in a flow of a liquid fuel.

### BACKGROUND

Bunkering, i.e. refuelling of ships, is estimated to be a US$ 125 billion industry. Fuel accounts for 60% of a ship's operating costs. Off-specification or contaminated fuel can adversely affect engine performance and cause serious damages. Such incidents are not uncommon, as over 100 ships a year are subject to such issues. To remove the contaminated fuel from the fuel tanks is typically costly, as the ship's operations need to be halted and professional equipment and personnel need to be brought on-board. With the International Maritime Organization (IMO) enforcing new compliance regulations on fuel, an off-specification fuel may be heavily fined. As fuel quality varies significantly in different regions or markets, improved fuel quality testing is imperative.

The current method of lab testing of fuel quality is based on a sampling method, and is manual, slow and prone to human and unforeseen errors. The test reports from this sampling method generally take 1-7 days to obtain, and until that time, the crew of the ship has no information about the fuel. Such delays are therefore costly and potentially disruptive.

It may be desirable to provide methods and devices for detecting contaminants in a liquid fuel that can address at least some of the above problems.

### SUMMARY

An aspect of the present disclosure provides a method of detecting at least one contaminant in a flow of a liquid fuel. The method comprises measuring one or more parameters of a flow of the liquid fuel; determining, based on the measured one or more parameters, one or more properties of the liquid fuel; extracting a plurality of features from selected ones of the one or more parameters and one or more properties; and applying a trained classification model on the extracted features to determine a type and a quantity of at least one contaminant in the liquid fuel.

The one or more parameters of the flow may be measured at a selected frequency, and the method further may comprise recording the one or more parameters of the flow and one or more properties of the liquid fuel as respective time series. Extracting the plurality of features may comprise running a feature window of a selected size over each of the selected ones of the time series.

The method may further comprise generating an alert if the quantity of the at least one contaminant is outside a predetermined range.

The one or more parameters of the flow may be selected from a group consisting of a differential pressure, a static pressure, a temperature and a mass flow rate. The one or more properties of the liquid fuel may comprise a density and a viscosity. The features may be selected from a group consisting of a slope, an amplitude, a wavelength, a frequency, a skewness, a magnitude, and a root mean square value. The at least one contaminant may be selected from a group consisting of water, sulphur, an oil, a gas, and a solid. The liquid fuel may comprise a fuel oil or a distillate.

The classification model may be trained based on a plurality of sets of labelled training data, and the labelled training data may comprise one or more of a liquid fuel with known properties, a liquid fuel with one or more known contaminants, and a flow with known parameters.

Another aspect of the present disclosure provides a system for detecting at least one contaminant in a flow of a liquid fuel. The system comprises a plurality of sensors configured to measure one or more parameters of a flow of the liquid fuel, and a processor. The processor is configured to determine, based on the measured one or more parameters, one or more properties of the liquid fuel; extract a plurality of features from selected ones of the one or more parameters and one or more properties; and apply a trained classification model on the extracted features to determine a type and a quantity of at least one contaminant in the liquid fuel.

The plurality of sensors may be configured to measure the one or more parameters of the flow at a selected frequency, and the processor may be further configured to record the one or more parameters of the flow and one or more properties of the liquid fuel as respective time series. The processor may be configured to run a feature window of a selected size over each of the selected ones of the time series to extract the plurality of features.

The processor may be further configured to generate an alert if the quantity of the at least one contaminant is outside a predetermined range.

The one or more parameters of the flow may be selected from a group consisting of a differential pressure, a static pressure, a temperature and a mass flow rate. The one or more properties of the liquid fuel may comprise a density and a viscosity. The features may be selected from a group consisting of a slope, an amplitude, a wavelength, a frequency, a skewness, a magnitude, and a root mean square value. The at least one contaminant may be selected from a group consisting of water, sulphur, an oil, a gas, and a solid. The liquid fuel may comprise a fuel oil or a distillate.

The classification model may be trained based on a plurality of sets of labelled training data, and the labelled training data may comprise one or more of a liquid fuel with known properties, a liquid fuel with one or more known contaminants, and a flow with known parameters.

Another aspect of the present disclosure provides a system for detecting at least one contaminant in a flow of a liquid fuel. The system includes a closed conduit through which the liquid fuel flows; a plurality of sensors disposed along the closed conduit; a mass flow meter connected to the closed conduit; and a processor communicatively coupled to the plurality of sensors and the mass flow meter. The processor is configured to monitor one or more parameters of the flow and one or more properties of the liquid fuel based on outputs from the plurality of sensors and the mass flow meter. The processor is configured to apply a trained classification model to determine a type and a quantity of at least one contaminant in the liquid fuel based on a change in features associated with the one or more parameters of the flow and one or more properties of the liquid fuel over a selected time period.

### BRIEF DESCRIPTION OF THE DRAWINGS

Embodiments of the disclosure will be better understood and readily apparent to one of ordinary skill in the art from the following written description, by way of example only, and in conjunction with the drawings, in which:

FIG. 1(a) shows a flow chart illustrating a method of detecting at least one contaminant in a flow of a liquid fuel according to an embodiment.

FIG. 1(b) shows a flow chart illustrating an implementation of the method of FIG. 1(a).

FIG. 2(a) show a block diagram of a system for detecting at least one contaminant in a flow of a liquid fuel according to an embodiment.

FIG. 2(b) shows a schematic diagram of an implementation of the system of FIG. 2(a).

FIG. 3(a) shows a model of a skid for testing and validating the method of FIG. 1(a) according to an embodiment.

FIG. 3(b) shows an enlarged view of a pipe segment of the skid of FIG. 3(a)

FIG. 4 shows a graph illustrating variation of oil-water mixture density with varying oil concentration.

FIG. 5 shows graphs illustrating changes in pressure drops with varying oil concentration at different flow velocities.

FIG. 6 shows a graph illustrating variation in drag coefficient with varying solid concentration in a flow.

FIG. 7, comprising 7(a)-7(c), shows results of a test of water flood in a fuel flow.

FIG. 8, comprising 8(a)-8(c), shows results of a test of water droplets in a fuel flow.

FIG. 9, comprising 9(a)-9(c), shows results of a test of heavy oil in a fuel flow.

FIG. 10, comprising 10(a)-10(c), shows results of a test of sand particles in a fuel flow.

FIG. 11, comprising 11(a)-11(b), shows results of a test of air injection in a fuel flow.

FIG. 12 shows a schematic block diagram of a computer system suitable for implementing at least some aspects of the present disclosure.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The present disclosure provides methods, devices and systems for detecting contaminants in a liquid fuel, during the transport process in pipelines. A liquid fuel of known properties is expected to exhibit certain flow characteristics. Contaminants in the liquid fuel usually change the properties of the fuel, and the flow characteristics of the contaminated fuel and the standard fuel would have differences. By picking up these variations in the flow parameters, it is possible to detect and classify the contaminant in the fuel. The disclosed technique utilizes standard flow instrumentation to capture the flow characteristics and then process them via software to detect and classify the contaminants.

In embodiments, a primary flow property that is utilized is the frictional gradient in the flow, metered using a differential pressure transducer. The frictional gradients are captured during flowing conditions. Changes in flow conditions affect the frictional gradient. Changes in temperature and static pressure also affect the frictional gradients. Hence, the flow instrumentation monitors the flow velocity utilizing a mass flow meter, temperature utilizing a temperature transducer, static pressure utilizing a pressure transducer. Changes in frictional gradients due to changes in flow velocity, temperature and static pressure are checked and accounted for.

The differential pressure readings are then processed and converted into viscosity. These algorithms form part of the pre-processing module. The viscosity signal is then analysed by a signal processing module to extract “features”, which are then inputted into the classification module. In the classification module, the features are cross-checked against training data to form a predictive estimate of the contaminant causing the anomaly in the viscosity signal. In some embodiments, the estimates are summarized for the full duration of the fuel transfer. In other words, the disclosed technique detects the contaminants in the flow as anomalies in the flow, captured by conventional flow instrumentation on the skid, and is then classified as contaminants.

Embodiments will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “inputting”, “calculating”, “determining”, “applying”, “extracting”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the scope of the disclosure.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G, 4G or 5G mobile telephone systems, as well as other wireless systems such as Bluetooth, ZigBee, Wi-Fi. The computer program when loaded and executed on such a computer effectively results in an apparatus that implements the steps of the preferred method.

The present disclosure may also be implemented as hardware elements. More particularly, in the hardware sense, an element is a functional hardware unit designed for use with other components or elements. For example, an element may be implemented using discrete electronic components, or it can form a portion of an entire electronic circuit such as an Application Specific Integrated Circuit (ASIC) or Field Programmable Gate Array (FPGA). Numerous other possibilities exist. Those skilled in the art will appreciate that the system can also be implemented as a combination of hardware and software elements.

According to various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which may be described in more detail herein may also be understood as a “circuit” in accordance with an alternative embodiment.

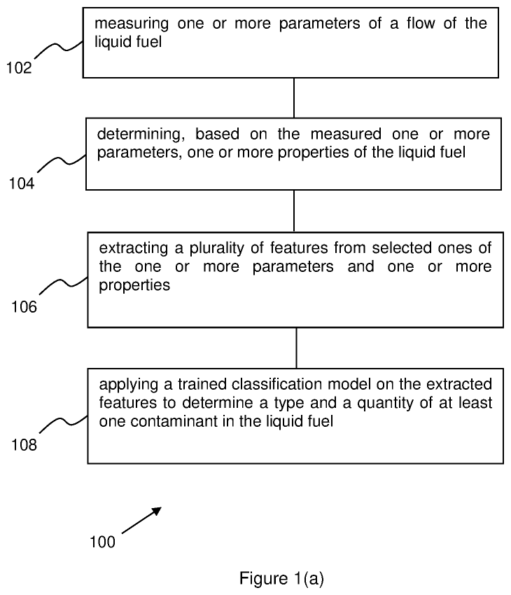

FIG. 1(a) shows a flow chart 100 illustrating a method of detecting at least one contaminant in a flow of a liquid fuel according to an embodiment. At step 102, one or more parameters of a flow of the liquid fuel are measured. At step 104, based on the measured one or more parameters, one or more properties of the liquid fuel are determined. At step 106, a plurality of features are extracted from selected ones of the one or more parameters and one or more properties. At step 108, a trained classification model is applied on the extracted features to determine a type and a quantity of at least one contaminant in the liquid fuel.

In some example implementations, thresholds of fuel quality are placed according to the relevant ISO standards for density, viscosity, water and solid content in the fuel. If the fuel quality is at an acceptable level although not ideal, the fuel transfer is allowed without any alarms. If the fuel quality is unacceptable (i.e. a quantity of a contaminant is outside an acceptable range), an alert is generated for necessary action.

The present method can monitor viscosity and contaminants, e.g. water content and solid content in the flow during the flow process itself (e.g. on the transfer pipeline). Such monitoring can facilitate online detection of anomalies in the fuel composition during the fuel transfer, rather than after the transfer from an offline fuel report from a laboratory test of a sample. The anomalies can be detected early, and appropriate action can be taken to prevent the usage of the off-specification fuel, reducing the risk of damage to ship's engine. By giving proof of good quality fuel transfer, the suppliers can avoid the risk of costly de-bunkering processes. Further, flow traceability is possible and a fuel data log of the entire fuel transfer can be recorded and used for legal paperwork if necessary.

FIG. 1(b) shows a flow chart 110 illustrating an example implementation of the method of FIG. 1(a) during a bunkering operation. At step 112, the estimated amount of fuel to be transferred is entered. At step 114, the acceptable contaminant amount in the fuel is entered for each type of contaminant, e.g. based on standard fuel grades or practical requirements. At step 116, analog signals from the measuring instruments, including those of the differential pressure, temperature, static pressure, and mass flow rate of the flow of the fuel, are received. At step 118, the received analog signals are converted into respective digital signals, for example, using analog-to-digital converters. At this stage, the signals may be recorded at step 120 as raw data for documentation in case they are needed at a later time, e.g. for analysis or for legal reasons. At step 122, a window block of the time series recording the signals is selected for analysis. At step 124, the signal to noise ratio (SNR) on the time series signals is monitored. This is done to ensure that there is sufficient signal intensity to detect features. At step 126, the signals are cross-correlated to check for dependencies. At step 128, the features are extracted from the time series signals. At step 130, anomalies [anomaly features] in the time series signals are detected against thresholds. At step 132, each detected anomaly is classified as a fuel contaminant by the classification model/engine. At step 134, the anomaly classification and SNR are logged for the window for documentation. In case a consolidate log of the entire operation is required, it can be generated at step 136. At step 138, the quantity of the off-specification fuel for the window is calculated (analogous to calculating the quantity of the contaminant). At step 140, the calculated quantity of contaminant is compared against the threshold initially set at step 114. If the quantity of contaminant is outside the acceptable range, an alarm/alert is raised at step 142 and logged. On the other hand, if the quantity of contaminant is within the acceptable range, the process repeats at step 122 for another window block.

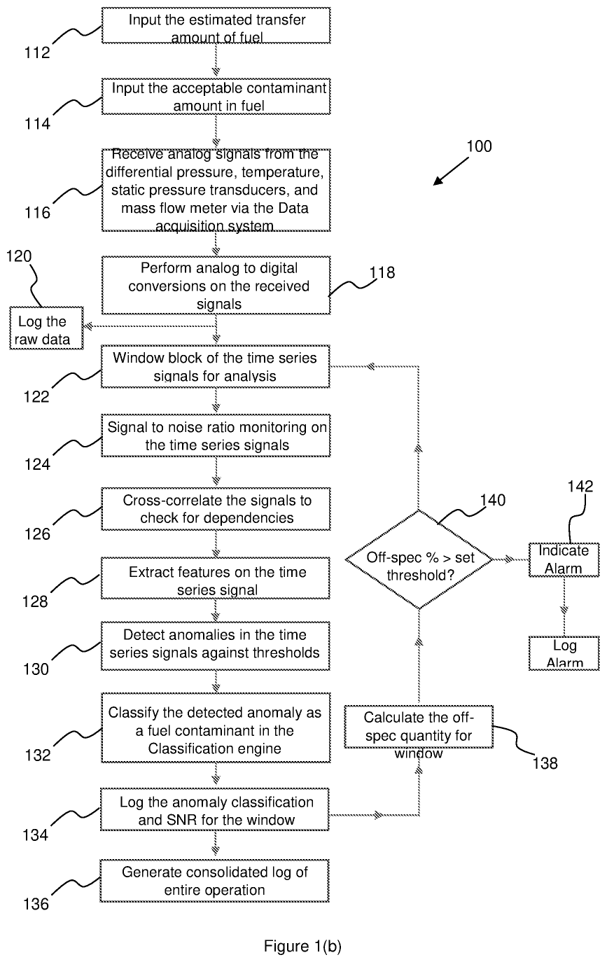

As described above, the present system includes both hardware instrumentation and a software component. FIG. 2(a) shows a general block diagram of a system 200 for detecting a contaminant in a liquid fuel in transit according to an embodiment. FIG. 2(b) shows a schematic diagram of an implementation of the system 200 of FIG. 2(a).

The hardware instrumentation can be implemented on a skid 202 which includes a pipe segment 204 with matching pipe diameter to that of the bunkering pipeline. This pipe segment 204 is fitted with a plurality of sensors 206 including a differential pressure transducer, temperature sensors, and a static pressure transducer. The differential pressure transducer (DPT) can measure the losses in pressure of the liquid fuel as it flows between two measuring points of the DPT. The measurement taken by the DPT is captured by a data acquisition system (DAQ) 212 as an analog signal. Similarly, live temperature measurements can be taken by the temperature sensors and inputted into the data acquisition system. Static pressure measurements can also be taken and inputted. Information regarding the density and velocity of the flow can be measured by a mass flow meter 208 on-board the bunkering vessel. The signals of the mass flow meter 208 are also logged into the DAQ 212. In one implementation, the information is logged at 10 Hz, or in other words, 10 measurements are taken every second.

The data acquisition system 212 acquires the analog signals from the instrumentation, digitizes and logs the data. The software component, which can be implemented by a computer system or processor 210, processes the information received from DAQ 212 as inputs and then calculates data such as the viscosity of the fluid, amount of water content and solid content in the fluid. This provides relevant information on the fluid properties, based on the flow. With reference to FIG. 2(a), a pre-processing module 216 extracts preliminary information about the contaminant detection and broadly classifies the contaminants. A feature extraction module 218 further acts upon this pre-processed data, as a refined detection tool, based on windowed operations on the pre-processed data. Further, these features are inputted into a classification module 220 to classify the contaminants.

In other words, the software component is built on a machine learning platform which can be trained to not only comprehend flow signatures of single phase oil, water, gas at various testing conditions but also comprehend and train the flow signatures on multiphase data. A multiphase flow may be defined as the co-current flow of immiscible phases (e.g. oil-water, air-water, oil-air, oil-sand, water-sand, or air-sand) through a single conduit.

The ability of the software component of the present system to comprehend multiphase data makes it applicable to fuel quality monitoring. When a fuel is contaminated, the secondary component in the flow may be another liquid or even a solid. This data can be extracted from the flow signatures by the software. By setting threshold limits to these contaminants, the user can have a bandwidth of fuel quality, with respect to contaminant quantity, which is acceptable to the user. When the thresholds are breached, an alert is sent to the user, e.g. via a user interface or display. Continuous deviations may cause an alarm to be sounded and prompt the user to take immediate action.

Referring to the sensors 206 in FIG. 2(b), the differential pressure transducer is configured to measure the pressure difference between the two points (i.e. tap point upstream tᵤₚ and tap point downstream t_{down}).

In one non-limiting example, the two tapping points are approximately 6.35 mm (1/4 inch) or 12.7 mm (1/2 inch) in diameter, drilled through the pipeline thickness, flush with the inner diameter of the pipe. The connections from the pipeline tapping points to the differential pressure's inputs are by stainless steel tubes, 8 mm in diameter, which connect as push-fit connectors. The differential pressure transducer has a bleed manifold that is used to ensure that the connection lines are filled with liquid.

The working principle of the differential pressure transducer is to measure the deflection of a sensing element due to the differential pressure between the two sides of the diaphragm. This deflection is quantified as an analog current or voltage signal by the transducer.

The chamber of the sensing element is typically liquid-filled. Therefore, the differential pressure transducer is normally mounted on the underside of the pipeline. The connectors are also filled with the same working fluid to avoid hydrostatic pressure head losses. The transducer has a 3-way manifold to facilitate bleeding of the connector lines. This can ensure that no air/gas bubbles are present in the connection lines, which may provide an erroneous reading. As gas is compressible, the air bubble can contract or expand depending on the pressure, which will lead to an inaccurate registration of pressure onto the diaphragm of the sensing element. Bleeding the connector lines can prevent air-entrapment.

Referring again to the sensors 206 FIG. 2(b), the temperature transducers in the present system can be contact-type temperature transducers to obtain temperature measurements. The measurement signals are sent by a resistance-type transmitter as 4-20 mA signals. The temperature transducers are typically mounted onto the pipeline on the 6.35 mm (1/4 inch) NPT thread tapping point. Static pressure on the line is monitored using static pressure transducer measuring gauge pressure on the pipeline. The static pressure transducer includes a sensor, e.g. a piezoresistive element, whose resistivity changes when pressure is applied upon the sensor. This is then converted into a 4-20 mA analog current signal. The static pressure transducer is mounted onto the pipeline on the 6.35 mm (1/4 inch) NPT thread tapping point. Further, to measure accurately the quantity of the liquid fuel flowing through the pipeline, a Coriolis mass flow meter is used.

In embodiments, the pre-processing module 216 of the software component (FIG. 2(a)) is utilized to normalize the data input. Due to this feature, the software can be scaled onto varying pipeline diameters, flow operating conditions and ambient conditions. The input data into the software includes

| | |
|---|---|
| 1. Differential pressure - | DP (Pascals) |
| 2. Temperature - | T (° C.) |
| 3. Static Pressure - | P (Pascals) |
| 4. Mass Flow rate - | $\dot{m} ( \frac{kg}{hr} )$ |
| 5. Density - | $ρ ( \frac{kg}{{m}^{3}} )$ |

Utilizing Hagen-Poiseuille equations, the differential pressure can be normalized into viscosity. Therefore, the input data can be normalized and reduced into fewer variables.

It has also been noted that viscosity of a fluid varies with temperature. Due to its temperature sensitivity, viscosity is normally mentioned with a reference temperature. The variations in viscosity measurements of marine fuel oil can be cross-checked and compensated for temperature variations by utilizing e.g. the ASTM D341 standards.

The signals captured form the instrumentation form a time series dataset, from which the different contaminants can be classified. There are alternate methods to classify a time series dataset. For example, one is to use a time series specific method whilst another is to extract features from the time series and to utilize supervised learning. In preferred embodiments, the latter method of extracting feature to pursue supervised learning is utilized. The signals, coming out of the pre-processing module 216, may display variations and these variations can then be linked to a specific contaminant dataset. This approach can facilitate more control onto the training of the machine learning models, onto a smaller dataset, than what would be required if the time series specific methods were to be employed.

The time-series data, obtained from the data acquisition system 212 is normalized into viscosity of the flow utilizing Hagen-Poiseuille equations as discussed above. In the feature extraction module 218, a feature window of a designated size is set and run throughout the signals. This can extract features such as slopes, amplitudes, wavelengths, frequencies, skewness, and other signal features for this window. Based on these features, the anomalies can be detected using the contaminant classification module 220.

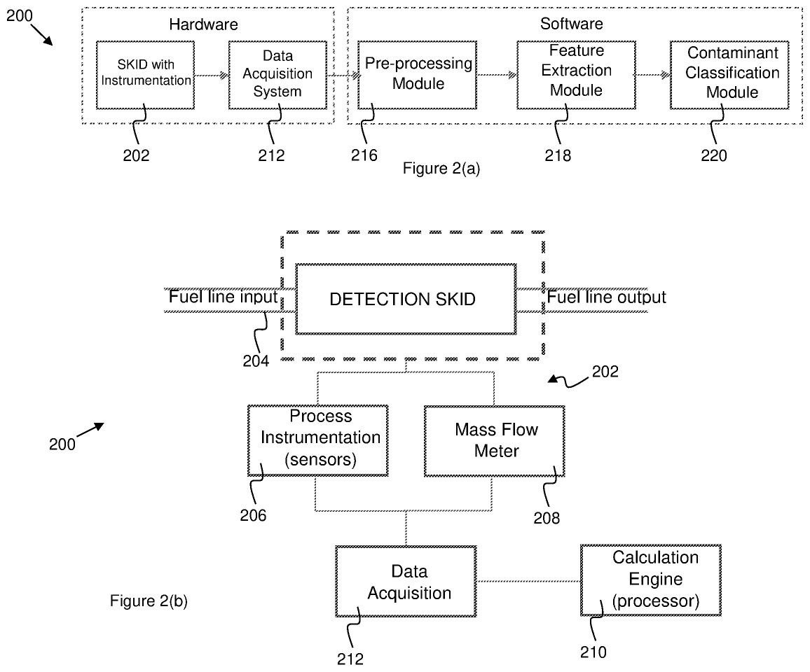

FIG. 3(a) shows a model of a skid 300 for testing and validating the method of FIG. 1(a) according to an embodiment. For example, scaled-down version of the skid 300 (i.e. a training skid) can be used to understand relationships and dependencies and to obtain data to train the classification model, while a full-size version of the skid 300 (i.e. a detection skid) can be used in practice to install onboard the vessels, according to the pipeline configuration of the vessel.

For example, the detection skid is installed onto the fuel transfer pipeline as shown in FIG. 2(b), forming part of the on-board fuel quality test unit which may also include a mass flow meter. The detection skid is based on a section of pipe identical to the fuel transfer line, installed adjacent (or inline) to the mass flow meter, onto the fuel transfer line. As described above, the instrumentation on the detection skid includes a differential pressure transducer, temperature transducers and a static pressure transducer, which are mounted onto the tapping points on the pipeline as shown in FIG. 2(b). The mass flow meter and the detection skid are connected to the pipeline via standard flanges. FIG. 3(b) shows an enlarged view of a pipe segment 302 of the skid 300 of FIG. 3(a). As can be seen in FIG. 3(b), a plurality of sensors, including a temperature sensor 304, a static pressure sensor 306, and a differential pressure sensor 308 with bleed manifold, are mounted along the pipe segment 302, which is a closed conduit that can be fitted to a transfer pipeline in use.

In one implementation, the signals acquired from the instrumentation are in the form of 4-20 mA analog current signals. These analog signals are captured from the instrumentation on the detection skid and the mass flow meter using a data acquisition system, e.g. a 12-bit data acquisition system that can acquire and digitize the signals at 10 Hz frequency. This raw data is stored separately as data files, before being inputting into the pre-processing module 216 (FIG. 2(a)).

In developing a training and testing regime, the relationships between different variables and parameters are analysed to identify various individual features that can be captured and interpreted.

First, the pressure drop can be captured using a differential pressure transducer as discussed above. The pressure drop is then represented as friction factors in the pipe.

The Reynolds number can be calculated represents a combination of parameters upon which the friction depends upon. By knowing the velocity, density and diameter of the pipe, the viscosity of the fluid can be determined. Hence from the pressure drop signature, viscosity of the fluid can be derived.

The viscosity of standard fuel oils is known through technical specifications, for example, as per the ISO 8217 specifications for marine fuel oils. The software component in the present system, being built of a machine learning platform, can be trained on the standard data for marine fuel oil, e.g. as per ISO 8217 standards for viscosity. Therefore, while testing the flow, the viscosity is calculated by the software component, and deviations from the standard fuel oil's viscosity can be registered as anomalies in the fluid composition.

Next, it is appreciated that the viscosity may change due to temperature difference. However, this parameter can be compensated for as per the ASTM D341 standard calculations. By measuring the temperature of the flow, via the temperature sensors (see FIGS. 2(b) and 3(b)), the corresponding live viscosity can be corrected based on ASTM D341.

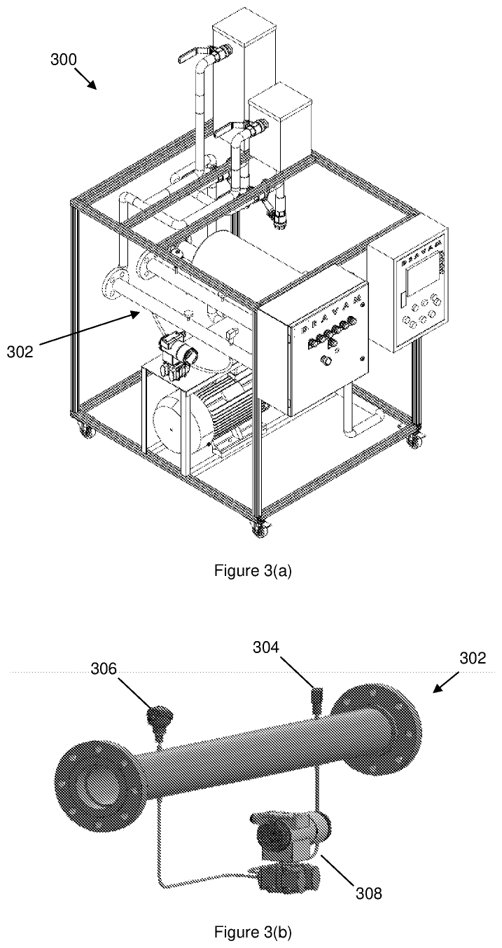

In addition, both the density and the viscosity of oils display changes due to changes in fluid composition. FIG. 4 shows a graph of changes in oil density when there is water contamination. When oil is contaminated with water (i.e. the ratio is lower than 1), the density of the oil is higher than that of pure oil (i.e. the ratio is 1). Utilizing this, dilatants added to marine fuel oil can be detected utilizing the changes in density based on technical specification such as the ISO 8217 standards. In practical implementations, the density can be monitored from the Coriolis mass flow meter.

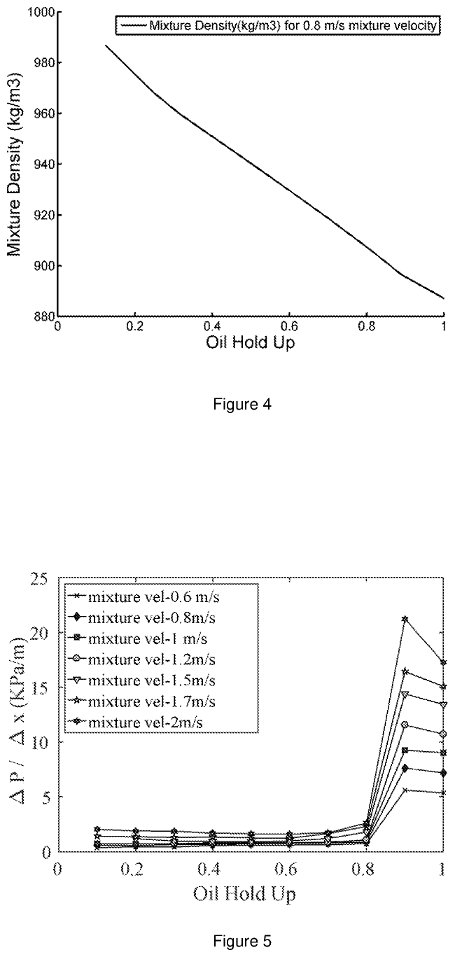

FIG. 5 shows the pressure drop per unit length plotted versus the oil hold up under different flow velocities, where a hold up value of 1 represents pure oil and 0 represents pure water. Generally, in marine fuel oils, low concentration of water may be found but this water can lead to a rise in viscosity and hence a larger flow resistance. Hence, deviations in the pressure signature can indicate higher concentration of water in the oil.

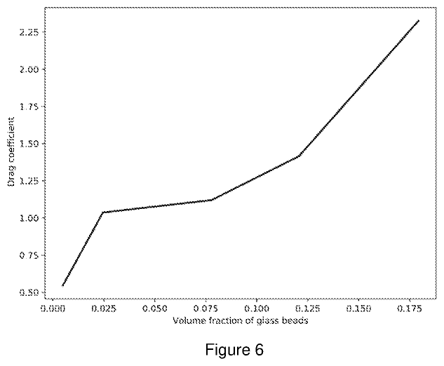

The presence of a solid in a liquid flow can also affect the pressure drop. FIG. 6 shows a graph of an example relationship between concentration of a solid in a flow and the resulting drag coefficients. As shown in FIG. 6, the presence of a solid in the flow has a significant influence on the frictional resistances in the pipeline and they generally co-relate with the concentration of the solid in the flow.

In embodiments, deliberate contamination of the fuel oil or distillate is used as part of training the algorithms to detect different features of the contaminated oil. For example, the skid 300 (FIG. 3) is used as a training skid for flow signature testing with various blends of marine fuel oils. The oil/distillate is sampled after passing through the training skid and then tested in a fuel testing laboratory to provide a constituent datasheet of the fuel sample. This datasheet can provide an accurate review of the contaminants in the fuel, conducted offline, on a small sample. This datasheet can then be utilized as a reference for the signals captured on the training skid. Preferably, a wide training set of input data is collected with measured variations to viscosity, density, water and solid content, to simulate all working conditions that are within the permissible limits of the ISO standards for fuel quality. Thresholds for the viscosity, density, water content and solid content are set as per requirements, such as international standards or widely-accepted specifications.

The testing is broadly classified as liquid-liquid flow testing, gas-liquid flow testing and solid-liquid flow testing, to broadly classify the contaminants as different phases, and further details are provided below in Examples 1-3.

### EXAMPLE 1

### Liquid-Liquid Flows

Liquid-liquid flows, with respect to fuel flows, can be sub categorized into oil-water flows, or high viscous oil—low viscous oil flows. Oil-water flows occur when the primary contaminant is water, which can occur as water flood or as water droplets in oil.

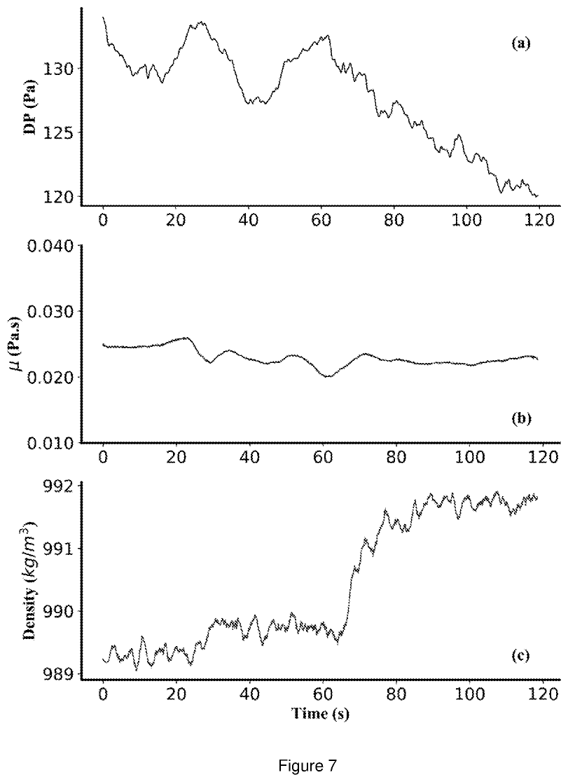

In order to simulate a water flood, whereby excess water flows with oil, the test skid is first run with oil and then flushed with water. The water is injected into the tank, to prevent any disturbance in flow rate in the main pipeline. The flow rate and other parameters are maintained as steady parameters. FIG. 7(a) represents the differential pressure signals, FIG. 7(b) represents the viscosity normalized signals, and FIG. 7(c) represents the density signals, during this water flood. The differential pressure readings are much lower than that of pure oil (in the range of 100 Pa, as opposed to 300-400 Pa for identical velocity). Also the reducing trend in viscosity can be noticed as a prominent feature. The viscosity of this mixture is lower than that of pure oil, due to mixing with water. Another prominent feature is the higher density readings in the mass flow meter, as can be seen in FIG. 7(c). As density of water is higher than oil, the Coriolis flow meter registers a higher reading than that of oil. Both these signal features are indicative of the occurrence of water flooding.

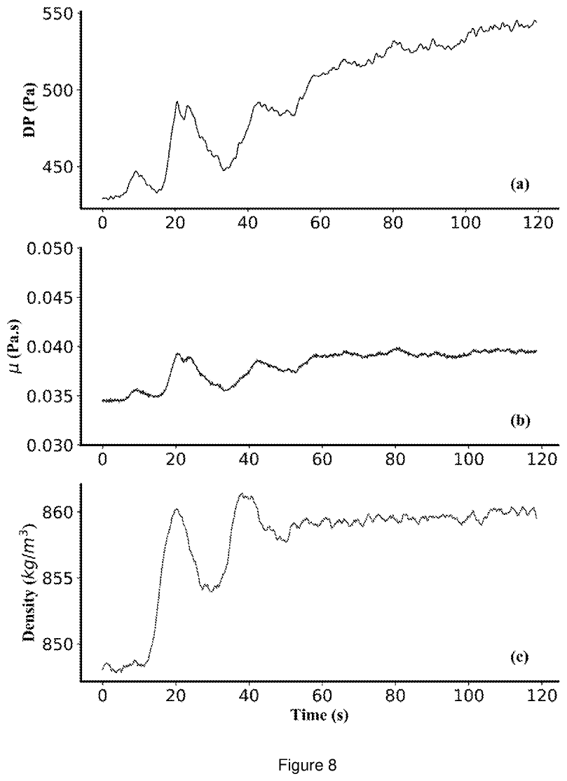

However, water can also occur as droplets in oil, rather than as large quantities. To simulate this in the testing skid, a specified amount of water is added to the oil in the tank, and accounts for between 0.5-1% of the fraction of oil in the tank and pipelines. When this flow is recoded over 2 minutes, FIG. 8(a) represents the differential pressure, FIG. 8(b) represents the viscosity of the mixture, and FIG. 8(c) represents the density signals that register.

As water is mixed with oil, the density of oil is expected to go up as shown by the density signal in FIG. 8(c). The average density also increases from around the 840 kg/m³ range to about 860 kg/m³. However, instead of reduction in differential pressure as was the case for water flooding, there is increment in differential pressure. This is due to the fact that water-in-oil, in lower quantities, leads to higher viscosity of the oil-water mixture in comparison to pure oil flow, as shown by the viscosity normalization in FIG. 8(b). This higher viscosity of the oil-water mixture leads to higher pressure drops in the pipeline.

The two sets of results in FIGS. 7(a)-(c) and FIGS. 8(a)-(c) show that utilizing signal features on the differential pressure signals and density signals, water contamination in oil can be detected and classified.

A high viscous oil—low viscous oil flow may include, for example, high viscosity fuel blends in fuel oil. Most marine fuels are blended with “cutter stocks”, which are basically different fuels, to attain the ISO 8217 specifications. In this process, at certain times, fuels with high variations in viscosity are used, which can lead to instability of fuels and subsequently lead to sludge formation. Sludge deposits in the fuel purifiers and fuel filters, and reduce the flow of fuel, effectively choking the fuel flow to the ship's engines. Sludge is a semi solid phase, having higher viscosity than the fuel itself.

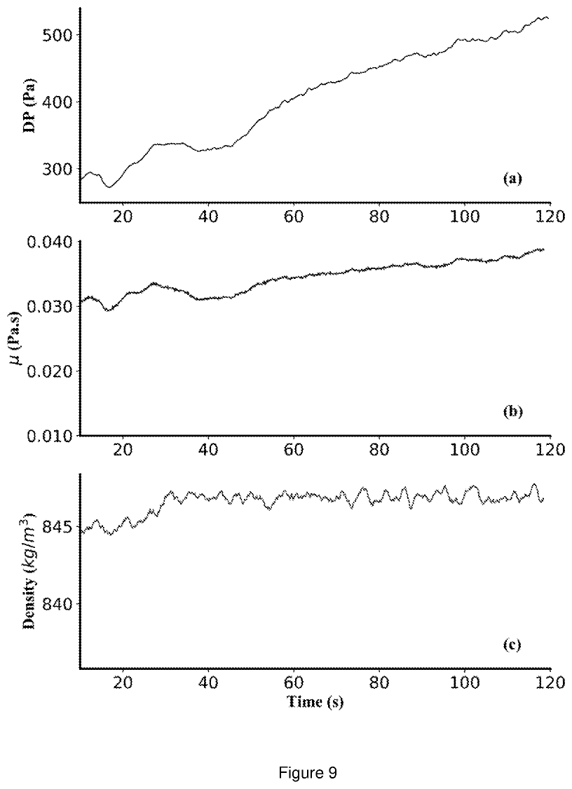

In an example test, a high-viscosity blend is added to the process oil, in small quantities, around 1% concentration, to simulate the flow of sludge in the fuel. FIG. 9(a) represents the differential pressure, FIG. 9(b) represents the viscosity of the mixture, and FIG. 9(c) represents the density signals from the test. The results show a steady rise in differential pressure, which then translates to a rise in viscosity. The high-viscosity oil being more dense than the fuel itself leads to higher density measurements by the mass flow meter as well. Both these signal change features can lend sufficient leverage to the feature extraction algorithm to extract the fact that the pattern change.

It will be appreciated that other types of high-viscosity contaminants, such as fuel with higher sulphur content, can be detected using a similar approach, as high-sulphur fuels generally display higher viscosity.

### EXAMPLE 2

### Solid-Liquid Flow

Solid particles may occur in fuel either as dirt, rust, metals or corrosion products in the pipeline. For example, marine fuel is generally treated and blended with catalysts, in a process known as cracking. Instances where poor filtration techniques are used can lead to such solid particles finding their way into the fuel and eventually into the ship's engine, where they can cause major damage. Therefore, it is desirable to detect solid particles in marine fuels.

In an example test, fine sand is to represent the solid particles in process oil. The solid particles are measured to approximately 0.5% phase fraction of the total fluid volume in the tank and pipeline, and added in the tank. No separate injection point is used, as any injection to the flow would give rise to a variation in the flow velocity which would affect the pressure drop signal from the transducer.

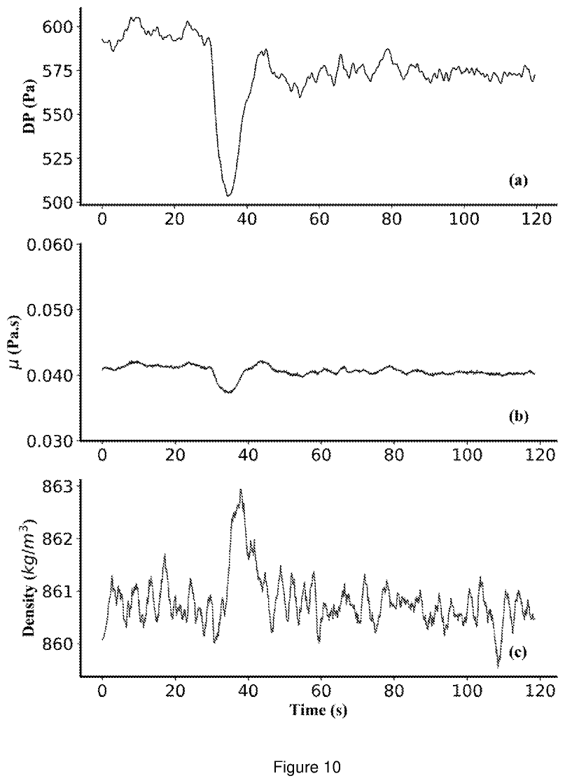

FIG. 10(a) represents the variations in differential pressure signals, FIG. 10(b) represents the variations in normalized viscosity of the flow, and FIG. 10(c) represents the density signals, captured for 2 minutes of flow time. As the solid particles pass through the pipeline, at the two tapping points, there is a variation in the differential pressure signal at around 40 seconds of the flow time. This variation is shown as a sharp dip in the pressure signal in FIG. 10(a). This occurs because the pressure at the upstream tapping point (tᵤₚ) increases first, which reduces the measured differential pressure AP. Subsequently, the differential pressure rises again as the solid particles pass through the downstream tapping point (t_{down})

It can also be seen that the viscosity is higher than that of pure oil, owing to higher resistance to flow due to the particles in the fluid. When a major cluster of solids pass through the two tapping points of the differential pressure transducer, the variation is also felt similar to the variation of the differential pressure.

Moreover, the density signal shows a sharp increase in density, owing to the higher density of the sand particles passing through the mass flow meter.

Both these signal change features can provide sufficient information to the feature extraction algorithm to extract the fact that the pattern change is caused by a solid contaminant that is passing through the pipeline.

### EXAMPLE 3

### Gas-Liquid Flow

Gas/air injection into fuel can cause the fuel's density to reduce, essentially translating to a smaller quantity of fuel being sold for price of a larger quantity. To test for air, on the testing skid, a separate air-injection line is installed, which can inject a designated amount of air into the fuel line, for a short duration of time. Utilizing this injection point, the test run is conducted to capture the changes in differential pressure and density from the Coriolis mass flow meter.

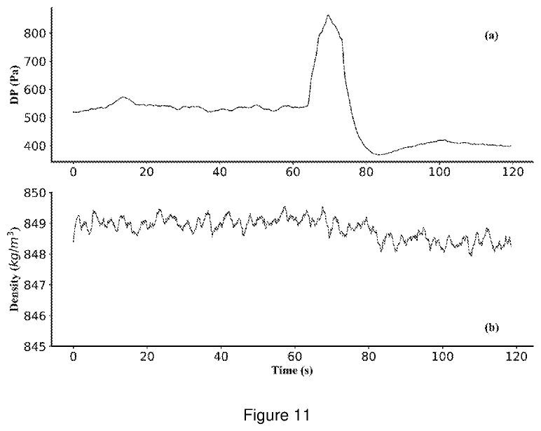

FIG. 11(a) represents the variations in the analog signals captured from the differential pressure transducer and FIG. 11(b) represents the density readings from the mass flow meter. During the point of air-injection, the differential pressure registers a sharp increase in the signal strength followed by a dip and eventual reduction. This happens because as air passes through the upstream pressure tapping point (tᵤₚ), there is a reduction in pressure at this point, which essentially increases the differential pressure sharply. As the air bubbles reach the downstream tapping point down), the pressures begin to equalize and the differential pressure is reduced. Eventually, the differential pressure is lower than that of pure oil. It will be appreciated that, in terms of frictional gradient, the dynamic viscosity of air is much lower than that of oil, or any liquid, which can explain why the differential pressure registers a lower reading.

The density shows a reduction though it is not as pronounced as the solids injection. This is because Coriolis mass flow meters typically do not function well with compressible fluids, as the instrument can only handle liquids and not gases.

Utilizing these two signals, it is possible to affirm the presence of air in the pipeline, as they form a distinct pattern in the signals from these two instruments.

From the above examples, it can be seen that various features can be extracted from the signals, and certain combinations of such features are indicative of a type of contaminant being present in the flow of the fuel. These patterns can be picked up by the classification model through training with large numbers of datasets. Example features of the time series signals that can be utilized include, but are not limited to, slope, amplitude (of crests and troughs), wavelength (of crest and troughs), frequency (via fast fourier transform), skewness, magnitude, and root mean square value.

Further, as each type of contaminants may have an indicative set of features, it is possible to simultaneously detect multiple types of contaminants in the flow over the same monitoring period. For example, from the differential pressure signals, one type of contaminant may be detected, and from the density signals, another type of contaminant may be detected. The classification model may be trained with large numbers of validation datasets (i.e. with the results validated against laboratory tests) to enhance the distinction of different contaminants.

In embodiments, the quantity of a certain contaminant can be estimated by amount of contaminant at a point of time at a cross-section (i.e. contaminant concentration) and the duration of the flow.

The contaminant concentration affects the magnitude of the signals, which is a feature on the signal, which is analyzed by the feature extraction module. For example, water as droplets shows a small contaminant concentration and shows a certain magnitude for density and viscosity signals. However, when larger concentrations are flowing through, the magnitudes of mixture viscosity and density change and the system can pick up the differences.

Once it is known that the varying contaminant concentration affects the signal magnitude, the system can be trained on more amounts of fuels with varying concentrations of the contaminants which can enable better accuracy in predicting the quantity of off-specification fuel.

In a window of flow (e.g. 5 minutes), knowing the mass flow rate during this time, and the proportion of the signals that has been classified as off-specification, it is possible to estimate the amount of the flow is off-specification (i.e. contaminated). As described above, the mass flow rate can be measured with the help of the mass flow meter on the skid. Generally, the flow rate is fixed for fuel transfer operations at 2 values, Qmin and Qmax, where Qmin is the low flow rate during the start and end of the bunker transfer and Qmax is the higher flow rate during the remaining times.

As described by utilizing flow instrumentation such as differential pressure transducer, temperature transducer, mass flow meter in example embodiments, it is possible to determine the density, viscosity, water and solid content in a flow of a liquid fuel by training the detection software, which is built on a machine learning platform. By training the detection software on standard marine fuels, with known acceptable grades, under varying flow conditions, it is possible to detect anomalies to these standards. This system can hence form a marine fuel monitoring system by detecting the contaminants in the flow, which are off-specification.

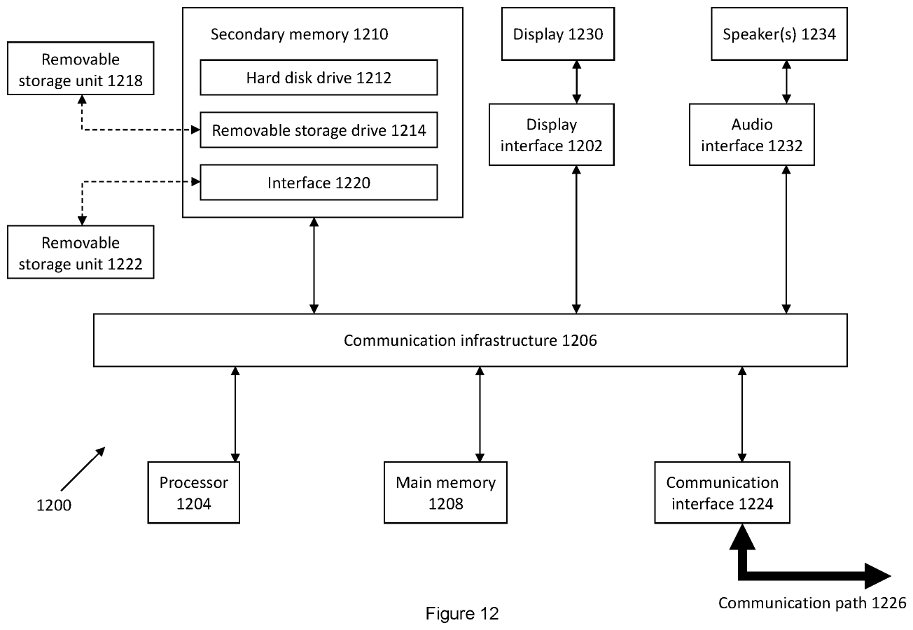

FIG. 12 depicts an exemplary computing device 1200, hereinafter interchangeably referred to as a computer system 1200, where one or more such computing devices 1200 may be used for implementing the software component. The following description of the computing device 1200 is provided by way of example only and is not intended to be limiting.

As shown in FIG. 12, the example computing device 1200 includes a processor 1204 for executing software routines. Although a single processor is shown for the sake of clarity, the computing device 1200 may also include a multi-processor system. The processor 1204 is connected to a communication infrastructure 1206 for communication with other components of the computing device 1200. The communication infrastructure 1206 may include, for example, a communications bus, cross-bar, or network.

The computing device 1200 further includes a main memory 1208, such as a random access memory (RAM), and a secondary memory 1210. The secondary memory 1210 may include, for example, a hard disk drive 1212 and/or a removable storage drive 1214, which may include a floppy disk drive, a magnetic tape drive, an optical disk drive, or the like. The removable storage drive 1214 reads from and/or writes to a removable storage unit 1218 in a well-known manner. The removable storage unit 1218 may include a floppy disk, magnetic tape, optical disk, or the like, which is read by and written to by removable storage drive 1214. As will be appreciated by persons skilled in the relevant art(s), the removable storage unit 1218 includes a computer readable storage medium having stored therein computer executable program code instructions and/or data.

In an alternative implementation, the secondary memory 1210 may additionally or alternatively include other similar means for allowing computer programs or other instructions to be loaded into the computing device 1200. Such means can include, for example, a removable storage unit 1222 and an interface 1220. Examples of a removable storage unit 1222 and interface 1220 include a program cartridge and cartridge interface (such as that found in video game console devices), a removable memory chip (such as an EPROM or PROM) and associated socket, and other removable storage units 1222 and interfaces 1220 which allow software and data to be transferred from the removable storage unit 1222 to the computer system 1200.

The computing device 1200 also includes at least one communication interface 1224. The communication interface 1224 allows software and data to be transferred between computing device 1200 and external devices via a communication path 1226. In various embodiments of the disclosure, the communication interface 1224 permits data to be transferred between the computing device 1200 and a data communication network, such as a public data or private data communication network. The communication interface 1224 may be used to exchange data between different computing devices 1200 which such computing devices 1200 form part an interconnected computer network. Examples of a communication interface 1224 can include a modem, a network interface (such as an Ethernet card), a communication port, an antenna with associated circuitry and the like. The communication interface 1224 may be wired or may be wireless. Software and data transferred via the communication interface 1224 are in the form of signals which can be electronic, electromagnetic, optical or other signals capable of being received by communication interface 1224. These signals are provided to the communication interface via the communication path 1226.

As shown in FIG. 12, the computing device 1200 further includes a display interface 1202 which performs operations for rendering images to an associated display 1230 and an audio interface 1232 for performing operations for playing audio content via associated speaker(s) 1234.

As used herein, the term “computer program product” may refer, in part, to removable storage unit 1218, removable storage unit 1222, a hard disk installed in hard disk drive 1212, or a carrier wave carrying software over communication path 1226 (wireless link or cable) to communication interface 1224. Computer readable storage media refers to any non-transitory tangible storage medium that provides recorded instructions and/or data to the computing device 1200 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 1200. Examples of transitory or non-tangible computer readable transmission media that may also participate in the provision of software, application programs, instructions and/or data to the computing device 1200 include radio or infra-red transmission channels as well as a network connection to another computer or networked device, and the Internet or Intranets including e-mail transmissions and information recorded on Websites and the like.

The computer programs (also called computer program code) are stored in main memory 1208 and/or secondary memory 1210. Computer programs can also be received via the communication interface 1224. Such computer programs, when executed, enable the computing device 1200 to perform one or more features of embodiments discussed herein. In various embodiments, the computer programs, when executed, enable the processor 1204 to perform features of the above-described embodiments. Accordingly, such computer programs represent controllers of the computer system 1200.

Software may be stored in a computer program product and loaded into the computing device 1200 using the removable storage drive 1214, the hard disk drive 1212, or the interface 1220. Alternatively, the computer program product may be downloaded to the computer system 1200 over the communications path 1226. The software, when executed by the processor 1204, causes the computing device 1200 to perform functions of embodiments described herein.

It is to be understood that the embodiment of FIG. 12 is presented merely by way of example. Therefore, in some embodiments one or more features of the computing device 1200 may be omitted. Also, in some embodiments, one or more features of the computing device 1200 may be combined together. Additionally, in some embodiments, one or more features of the computing device 1200 may be split into one or more component parts.

It will be appreciated that the elements illustrated in FIG. 12 function to provide means for performing the various functions and operations of the servers as described in the above embodiments.

In an implementation, a server may be generally described as a physical device comprising at least one processor and at least one memory including computer program code. The at least one memory and the computer program code are configured to, with the at least one processor, cause the physical device to perform the requisite operations.

It will be appreciated by a person skilled in the art that numerous variations and/or modifications may be made to the present disclosure as shown in the specific embodiments without departing from the scope of the disclosure as broadly described. For example, different combinations of parameters and properties may be used depending on the type of fuel in the flow (e.g. fuel oil, distillate, etc.). The detection window may also be varied. The present embodiments are, therefore, to be considered in all respects to be illustrative and not restrictive.

1. A method of detecting at least one contaminant in a flow of a liquid fuel within a pipeline, the method comprising:
measuring one or more parameters of a flow of the liquid fuel between two points on the pipeline;
determining, based on the measured one or more parameters, one or more properties of the liquid fuel, and thereby establishing a correlation between the flow parameters and the fuel properties;
extracting a plurality of features from selected ones of the one or more parameters and the one or more properties of the liquid fuel, wherein the features are associated with a change in one or more said properties; and
applying a trained classification model on the extracted features to determine a type and a quantity of at least one contaminant in the liquid fuel.

2. The method according to claim 1, wherein the one or more parameters of the flow are measured at a selected frequency, wherein the method further comprises recording the one or more parameters of the flow and one or more properties of the liquid fuel as respective time series, and wherein extracting the plurality of features comprises running a feature window of a selected size over each of the selected ones of the time series.

3. (canceled)

4. (canceled)

5. The method according to claim 1, wherein the one or more parameters of the flow are selected from a group consisting of a differential pressure, a static pressure, a temperature and a mass flow rate.

6. The method according to claim 1, wherein the one or more properties of the liquid fuel comprise a density and a viscosity.

7. The method according to claim 2, wherein the features are selected from a group consisting of a slope, an amplitude, a wavelength, a frequency, a skewness, a magnitude, and a root mean square value.

8. The method according to claim 1, wherein the at least one contaminant is selected from a group consisting of water, sulphur, an oil, a gas, and a solid, the method further comprising generating an alert if the quantity of the at least one contaminant is outside a predetermined range.

9. The method according to claim 1, wherein the liquid fuel comprises a fuel oil or a distillate.

10. The method according to claim 1, wherein the classification model is trained based on a plurality of sets of labelled training data, and wherein the labelled training data comprises one or more of a liquid fuel with known properties, a liquid fuel with one or more known contaminants, and a flow with known parameters.

11. The method according to claim 1, wherein extracting the features comprises applying a machine learning platform, and wherein the machine learning platform is trained to comprehend multiphase flow data.

12. A system for detecting at least one contaminant in a flow of a liquid fuel within a pipeline, the system comprising:
a plurality of sensors configured to measure one or more parameters of a flow of the liquid fuel between two points on the pipeline; and
a processor configured to:
determine, based on the measured one or more parameters, one or more properties of the liquid fuel, and thereby establishing a correlation between the flow parameters and the fuel properties;
extract a plurality of features from selected ones of the one or more parameters and the one or more properties of the liquid fuel, wherein the features are associated with a change in one or more said properties; and
apply a trained classification model on the extracted features to determine a type and a quantity of at least one contaminant in the liquid fuel.

13. The system according to claim 12, wherein the plurality of sensors are configured to measure the one or more parameters of the flow at a selected frequency, wherein the processor is further configured to record the one or more parameters of the flow and one or more properties of the liquid fuel as respective time series, and wherein the processor is configured to run a feature window of a selected size over each of the selected ones of the time series to extract the plurality of features.

14. (canceled)

15. The system according to clam 12, wherein the processor is further configured to generate an alert if the quantity of the at least one contaminant is outside a predetermined range.

16. The system according to claim 12, wherein the one or more parameters of the flow are selected from a group consisting of a differential pressure, a static pressure, a temperature and a mass flow rate.

17. The system according to claim 12, wherein the one or more properties of the liquid fuel comprise a density and a viscosity.

18. The system according to claim 13, wherein the features are selected from a group consisting of a slope, an amplitude, a wavelength, a frequency, a skewness, a magnitude, and a root mean square value.

19. The system according to claim 12, wherein the at least one contaminant is selected from a group consisting of water, sulphur, an oil, a gas, and a solid.

20. The system according to claim 12, wherein the liquid fuel comprises a fuel oil or a distillate.

21. The system according to claim 12, wherein the classification model is trained based on a plurality of sets of labelled training data, and wherein the labelled training data comprises one or more of a liquid fuel with known properties, a liquid fuel with one or more known contaminants, and a flow with known parameters.

22. The system according to claim 12, wherein the processor is configured to extract the features based on a machine learning platform, and wherein the machine learning platform is trained to comprehend multiphase flow data.

23. A system for detecting at least one contaminant in a flow of a liquid fuel within a pipeline, the system comprising:
a closed conduit through which the liquid fuel flows;
a plurality of sensors disposed along the closed conduit;
a mass flow meter connected to the closed conduit; and
a processor communicatively coupled to the plurality of sensors and the mass flow meter,
wherein the processor configured to monitor one or more parameters of the flow between two points on the pipeline and one or more properties of the liquid fuel based on outputs from the plurality of sensors and the mass flow meter, and
wherein the processor is configured to apply a trained classification model to determine a type and a quantity of at least one contaminant in the liquid fuel based on features associated with a change in the one or more parameters of the flow and the one or more properties of the liquid fuel over a selected time period.
